(19) United States
(12) Patent Application Publication
(10) Pub. No.: US 2019/0282369 A1
(43) Pub. Date: Sep. 19, 2019

(54) CONSTRAINED PROSTHESIS FOR THE KNEE

(71) Applicant: TECRES S.p.A., Sommacampagna (Verona) (IT)

(72) Inventors: Giovanni FACCIOLI, Monzambano (MN) (IT); Renzo SOFFIATTI, Nogara (VR) (IT)

(73) Assignee: TECRES S.p.A., Sommacampagna (Verona) (IT)

(21) Appl. No.: 16/301,183

(22) PCT Filed: May 10, 2017
(86) PCT No.: PCT/IB2017/052734

§ 371 Date: Nov. 13, 2018

(30) Foreign Application Priority Data
May 16, 2016 (IT) 102016000050133

(51) Int. Cl. A61F 2/38

(52) U.S. Cl. CPC A61F 2/3836; A61F 2/389; A61F 2/3859

(57) ABSTRACT

Prosthesis for the knee joint including a tibial component, adapted to be fixed to one end of the tibial bone in proximity to the knee joint, and a femoral component, adapted to be fixed to one end of the femoral bone in proximity to the knee joint, wherein the tibial component is adapted to come into contact and be articulated with the femoral component, wherein the tibial component includes a protrusion adapted to be inserted in an opening present in the femoral component; method for the assembly of the prosthesis.

### TECHNICAL FIELD OF THE INVENTION

The present invention refers to a constrained joint prosthesis adapted to be implanted at a knee joint. A joint prosthesis is intended to be implanted for a final use and hence its duration is permanent—except if particular drawbacks arise or if there is an onset of infection, i.e. except for very particular and specific cases.

### DESCRIPTION OF RELATED ART

As is known, joint prostheses are implanted in the human body at a bone or at a bone articulation that is damaged or weakened.

A joint prosthesis is intended to be implanted in a definitive manner and hence its duration is permanent, except for very particular and specific cases in which the prosthesis is removed.

When the implant site is particularly weak or damaged, or when the implant of a prosthesis occurs for a second time, for example due to the substitution of a first prosthesis that is infected, it is necessary to use a constrained prosthesis, e.g. re-implant prosthesis, in which its femoral and tibial components are connected or constrained to each other.

It is therefore necessary to arrange a preformed constrained prosthesis, capable of providing the aforesaid constraint between the components thereof.

### SUMMARY OF THE INVENTION

One object of the present invention is to improve the state of the prior art.

A further object of the present invention is to provide a constrained prosthesis, also possibly secondary or re-implant prosthesis, which is preformed and simultaneously able to allow good mobility for the patient, notwithstanding the weakness of the tissues surrounding the implant.

A further object of the present invention is to provide a constrained prosthesis that ensures high stability for the knee joint.

In accordance with one aspect of the present invention, a constrained prosthesis for a knee is provided according to the present application.

The present invention further refers to a method for the assembly of a constrained prosthesis for a knee according to the present application.

The present application refers to preferred and advantageous embodiments of the invention.

### BRIEF DESCRIPTION OF THE DRAWINGS

Further characteristics and advantages of the present invention will be more evident from the detailed description of a preferred but not exclusive embodiment of a constrained prosthesis for a knee, illustrated as a non-limiting example in the enclosed drawing tables in which:

FIG. 1 and FIG. 2 are perspective views of the constrained prosthesis according to the present invention which illustrate two mutual positions of a femoral component with respect to a tibial component;

FIGS. 3 to 5 are respectively a front view, a top view and a side view of the tibial component of the constrained prosthesis according to the present invention;

FIGS. 6 to 9 are respectively a side perspective view, a top view, a bottom front view and a bottom rear view of the femoral component of the constrained prosthesis according to the present invention;

FIGS. 10 and 11 are front views of the constrained prosthesis according to the present invention respectively provided or not provided with a further closure or locking element;

FIGS. 12 and 13 are side schematic views of the constrained prosthesis according to the present invention which illustrate two mutual positions of the femoral component with respect to the tibial component;

FIGS. 14 and 15 are side schematic views in section of the constrained prosthesis according to the present invention pursuant to FIGS. 12 and 13, taken along the trace plane XIV-XIV of FIG. 10;

FIG. 16 is a side perspective view of the constrained prosthesis according to one version of the invention; and

FIG. 17 is an exploded side perspective view of the constrained prosthesis according to one version of the invention.

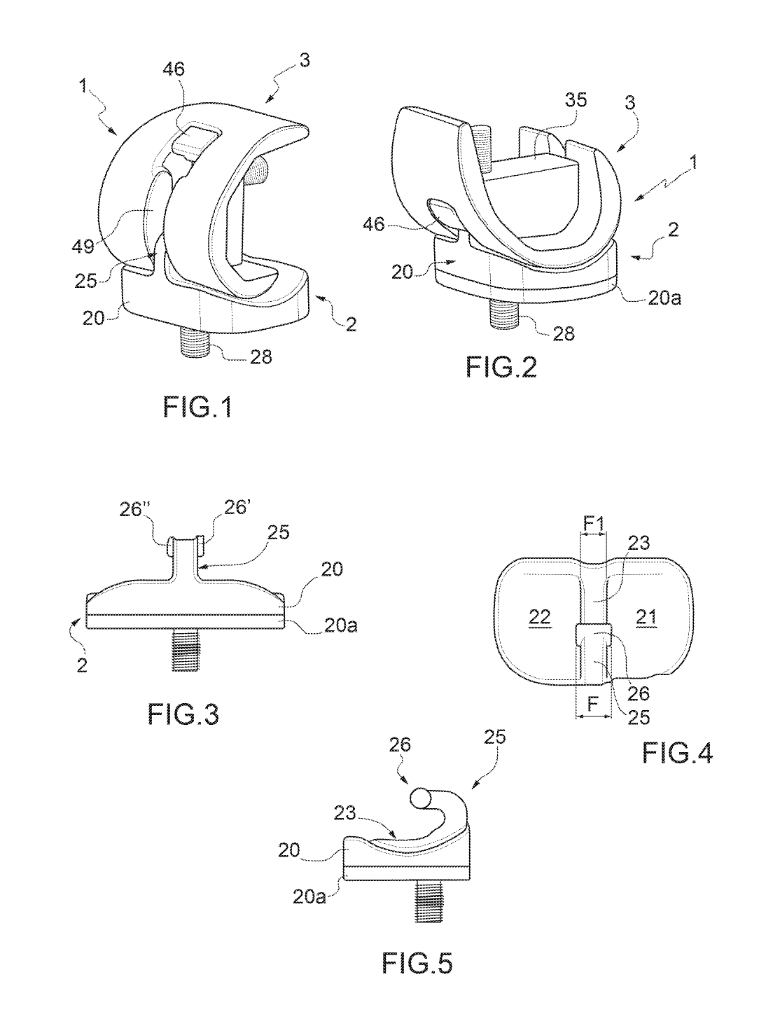

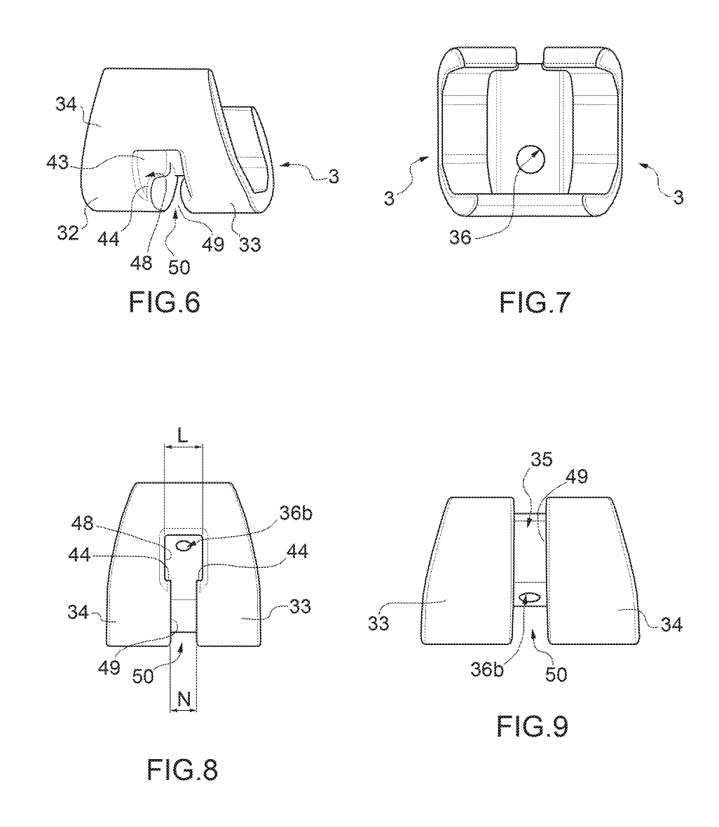

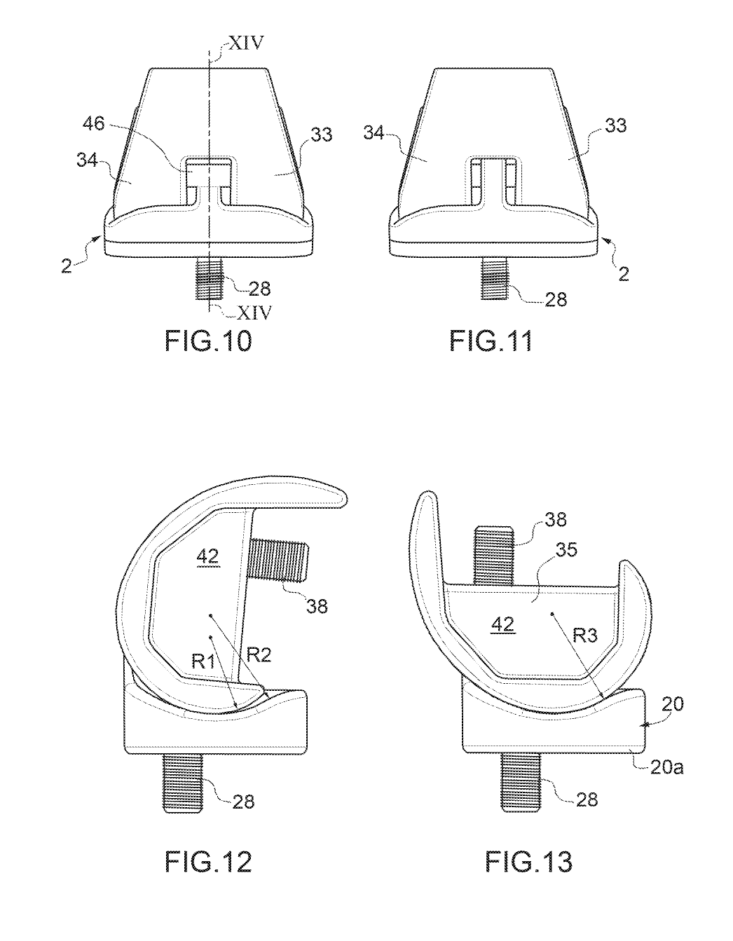

### DETAILED DESCRIPTION OF THE INVENTION

With reference to the enclosed figures, reference number 1 generically indicates a constrained prosthesis.

By constrained prosthesis, it is intended a prosthesis whose tibial and femoral components are joined together, i.e. they are hinged or it is not possible to modify the relative distance therebetween.

In substance, the femoral component is capable of rotating and translating with respect to the tibial component (and vice versa) but the two components cannot be separated from each other. This serves so that, once the two components are fixed to the bone of the patient during implant, the knee joint cannot undergo dislocations, or that tibia and femur do not have irregular relative rotation.

The femoral component and the tibial component of the prosthesis 1 according to the present invention, according to that defined now and as will be better understood in the course of the present description, are constrained to each other in an articulated manner, and they are both adapted to be stably and/or permanently fixed to the respective bone of the patient.

Such constrained prosthesis is preformed and adapted to be implanted at the knee joint.

The constrained prosthesis 1 according to the present invention comprises a tibial component 2 and a femoral component 3.

The tibial component 2 is adapted to be fixed to the end of the tibial bone at the knee joint while the femoral component 3 is adapted to be fixed to the end of the femoral bone in proximity to the knee joint.

In one version of the invention, only the femoral component 3 and the tibial component 2 are present, constrained to each other, while no patellar component is present.

The tibial component 2 comprises a tibial plate 20, on which the femoral component 3 is adapted to abut, roll and slide and with which it is articulated.

In one version of the invention, the tibial plate 20 abuts against and/or is fixed to a tibial base 20a which is adapted to come into contact during use with the tibial bone of the patient.

The tibial plate 20 and the tibial base 20a constitute, at least in one version of the invention, the tibial component 2 of the prosthesis 1 according to the present invention.

The tibial plate 20 comprises two condylar articular bases 21, 22.

The condylar articular bases 21, 22 are substantially concave and have a curvature radius R2 (as is visible in FIG. 12).

The tibial component 2 further comprises, between the condylar articular bases 21, 22, a rib 23.

Such rib 23 is in relief with respect to the surface of the tibial plate 20 and/or of the condylar articular bases 21, 22.

At its front portion, the tibial component 2 and/or the rib 23 has a protrusion 25. Such protrusion 25 has a substantially curved and/or C-shaped or swan-neck projection and during use is projected upward or towards the femoral component 3. The protrusion 25 has a head or end 26 that is substantially T-shaped.

Specifically, the protrusion 25 comprises, as stated, a substantially curved shape and/or C shape comprising a first section that corresponds with at least part of the rib 23, a second section, with substantially vertical section, and a third section, raised with respect to the rib 23, but which is substantially parallel to the latter.

Such third section departs from the second section and terminates with the head or end 26, shaped as a T.

In one version of the invention, the rib 23 is not present and therefore the protrusion 25 only has a section with substantially vertical progression, from which a further section departs, substantially parallel to the tibial plate 20 and which terminates with the head or end 26.

The third section or the further section of the protrusion 25 is extended towards the rear part of the tibial component 2.

Therefore, the head or end 26 of the protrusion 25 comprises two sections 26′, 26″ that are extended on opposite sides with respect to the third section (and/or to the protrusion 25). In one version of the invention, the two sections 26′, 26″ are lateral bulges of the protrusion 25 or of its third section.

The sections 26′, 26″ have a progression that is lateromedial or parallel to the transverse axis of the human body.

The transverse axis of the human body is an axis of the human body that goes from right to left.

Due to the shape of the protrusion 25, the head or end 26 thereof comes to be situated in raised position substantially at the center of the tibial plate 20 and/or of the rib 23.

In particular, as visible in FIG. 15, the head or end 26 is situated at a distance D from the rib 23 or at a distance B from the most-front part of the tibial plate 20 and/or of the tibial component 2.

As visible in FIG. 4, the head or end 26, including the sections 26′, 26″, has a size or width, calculated according to the transverse axis of the human body, equal to F.

In particular, F is greater than the width F1 of the rib 23 (and/or of the protrusion 25).

The width of the first and/or second and/or third section or of the section with substantially vertical progression and/or of the further section of the protrusion 25 also have a width substantially corresponding to F1.

The tibial component 2 and/or the tibial base 20a, along a surface adapted to come into contact with the tibial bone of the patient or in any case opposite the condylar articular bases 21, 22, has a hole 24.

The hole 24 is adapted to house and fix, through for example a screw-nut screw coupling, at least one pin 28.

The at least one pin 28, possibly threaded, is a pin adapted for the connection, fixing and/or centering or orientation of the tibial component 2 with respect to the bone end of the tibial bone of the patient.

The at least one pin 28 is extended from the face of the tibial component 2 opposite that which has the two condylar articular bases 21, 22 or from the lower during use face of the tibial base 20a.

In one version of the invention, the at least one pin 28 is adapted to be screwed into a suitably threaded hole of a stem 28a (or to be connected to the same in a suitable manner), adapted for the connection, fixing and/or centering or orientation of the tibial component 2 with respect to the bone end of the tibial bone of the patient.

The protrusion 25, during use, is adapted to be inserted in the femoral component 3, in a manner such to create a guided articulation or a constraint articulation between the femoral component 3 and the tibial component 2.

The femoral component 3 has a shape that generally reproduces the condylar articulation surfaces of the femur.

In particular, the femoral component 3 has, as is visible for example in FIGS. 12 and 13, a substantially U-shaped form—in cross section according to a plane parallel to the sagittal plane of the human body.

The femoral component 3 comprises an internal surface 31, substantially concave, adapted to be positioned in contact with the bone seat, and an external surface 32, substantially convex, adapted to come into contact and be articulated with the tibial component 2 and/or with the tibial plate 20.

The femoral component 3 is symmetric, with respect to a plane of symmetry parallel to the sagittal plane of the human body.

More in detail, the femoral component 3 comprises a first condylar portion 33 placed laterally and a second condylar portion 34 medially placed with respect to the sagittal plane of the human body, each having a shape similar to that of the condyles of the knee.

The condylar portions 33, 34 in turn have a substantially U-shaped form—in cross section according to a plane parallel to the sagittal plane of the human body.

The condylar portions 33, 34, in their front during use portion of the constrained prosthesis 1 according to one version of the present invention, converge towards each other and are joined together to form a joining portion or surface adapted to come into contact with the patella or to be arranged substantially in the zone where it is usually situated from the anatomical standpoint.

The condylar portions 33, 34 are during use adapted to come into contact with and be articulated on the condylar articular bases 21, 22 of the tibial component 2.

The condylar portions 33, 34 are separated from each other by an intercondylar space 50. The intercondylar space 50 is extended at least along the during use rear and central portion of the femoral component 3 and/or of the condylar portions 33, 34. In one version of the invention, the intercondylar space 50 is also extended for a portion of the front part of the femoral component 3, e.g. the portion which, during use, is placed downward and towards the central portion thereof.

The femoral component 3 comprises a box element 35, placed on the internal surface 31.

The box element 35 is connected to the internal surface 31 and, in one version of the invention, is integrally made therewith and/or with the femoral component 3.

The box element 35 has two lateral walls and one connector wall, which connects and joins the lateral walls. The connector wall is placed above the lateral walls of the box element. The lateral walls have a progression parallel to the sagittal plane of the human body. During use, they are extended therefore from the front portion to the rear portion of the internal surface 31 of the tibial component 3.

The box element 35 has a substantially overturned U shape—in cross section according to a plane parallel to the front plane of the human body.

Between the lateral walls and the connector wall of the box element 35, a seat or cavity is comprised or enclosed, corresponding to the abovementioned intercondylar space 50.

At the connector wall of the box element 35, a hole 36 and/or a cylindrical protrusion or stem 38 (possibly threaded) can be present, for the connection, fixing and/or centering or orientation of the femoral component 3 with respect to the bone end of the femoral bone.

In one version of the invention, the at least one pin 38 is adapted to be screwed in a suitably threaded hole of a stem 38a (or to be connected to the same in a suitable manner), adapted for the connection, fixing and/or centering or orientation of the tibial component 2 with respect to the bone end of the tibial bone of the patient.

The condylar portions 33, 34 have, in at least one portion of their profile, a curvature radius R1, as is visible for example in FIG. 12.

The radius R1 is present at least in the rear portion of the condylar portions 33, 34.

In a further version of the invention, the condylar portions 33, 34 have, in at least one portion of their profile, a curvature radius R3, as is visible for example in FIG. 13.

The radius R3 is present at least in the central portion of the condylar portions 33, 34.

The curvature radius R1, in one version of the invention, is smaller than the curvature radius R2.

In a further version, the curvature radius R3 is substantially equal to the curvature radius R2.

The box element 35 comprises an external surface 42 and an internal surface 43. The internal surface 43 faces towards the intercondylar space 50.

The internal surface 43 in turn comprises two internal lateral walls and one internal connector wall, which delimit the intercondylar space 50.

Each or at least one of the internal lateral walls of the internal surface 43 have a step 44. Such step 44 is projecting with respect to the internal lateral surface of the internal surface 43. Therefore, the step 44 is extended towards the intercondylar space 50.

Such step 44 constitutes a kind of guide for the head or end 26 of the tibial component 2. Indeed, such head or end 26, during the articulation of the femoral component 3 on the tibial component 2, slides and/or can rotate along such step 44, allowing the articulation of the knee of the patient in which the constrained prosthesis 1 has been implanted.

Simultaneously, the step 44 determines the constraint of the femoral component 3 with the tibial component 2, since the head or end 26 of the latter abuts against (particularly due to the sections 26′, 26″) and is substantially prevented—regarding its movements from right to left—from exiting from the seat determined by the box element 35 and/or, hence, from the intercondylar space 50.

In one version of the invention, such step 44 has a substantially overturned S shape.

Regarding such overturned S, during use: the upper horizontal section coincides with the internal connector wall, the concavity is placed at the rear part of the tibial component 3, the convexity is placed at the front part of the tibial component 3 and the lower horizontal second section coincides with the (central) external surface 42 of the femoral component 3.

The overturned S shape of the step 44 and/or the presence of the step 44 allows creating an opening, in the femoral component 3 and in particular between its two condylar portions 33, 34, comprising two portions and/or having two widths. By width, in this case, it is intended the size of the opening along a direction that goes from right to left, hence considered along the front plane of the human body or parallel to the same. Such direction is horizontal, i.e. also parallel to the abutment surface of the human body.

The opening determined by the box element 35 and by the step 44 coincides with the intercondylar space 50.

In particular, as is visible for example in FIG. 8, for example in proximity to the front zone of the femoral component 3, a first opening 48 is present having a width L.

In proximity instead to the central and lower zone of the femoral component 3, a second opening 49 is present which has a width N, in which N is smaller than L. In particular, as will be better described hereinbelow, the width L corresponds substantially or is slightly greater than the width F of the head or end 26 of the femoral component 2. In such a manner, it is possible to insert such head or end 26 inside the intercondylar space 50, through such first opening 48.

Then, once the protrusion 25 is inserted in the femoral component 3 by means of its head or end 26, the latter is engaged with, or at least abutted against, the step 44 and is unable to exit from the second opening 49 having width N.

Indeed, the width N is smaller than the width F and substantially corresponds to the width F1, or is slightly greater than the latter.

According to a section taken along a plane parallel to the front plane of the human body, therefore, the opening has a substantially T shape, substantially corresponding to the T shape of the protrusion 25 and/or of the head or end 26 of the protrusion 25.

The constrained prosthesis 1, according to at least one version of the invention, then comprises a closure or locking element 46.

The closure or locking element 46 has size substantially corresponding to that of the first opening 48 or at least of its initial part. In particular, such closure or locking element 46 is capable of closing the first opening 48 and preventing the exit of the head or end 26 from the intercondylar space 50 delimited by the box component 35 of the femoral component 3.

In one version of the invention, the closure or locking element 46 is provided with a pin 47, adapted to be inserted, e.g. snap-inserted, in at least one hole 36b suitably made in the femoral component 3.

The hole 36b is placed at the front portion of the femoral component 3, in a manner so as to maintain the closure or locking element 46 in the front or frontal portion thereof.

In a further version of the invention, not illustrated in the figures, closure or locking element 46 comprises a through hole. The longitudinal progression of such through hole can correspond with the longitudinal progression of the hole 36b present in the femoral component 3.

For example, in one version of the invention, such holes can have a substantially vertical longitudinal progression, considering the prosthesis according to the present invention in its implant position or during use.

In this version, the pin 47 is not present and is substituted by a screw, for example a metallic screw. Such screw passes, possibly being screwed, through the longitudinal hole of the closure or locking element 46 and, possibly, also through the hole 36b.

In such a manner, the locking (from the outside, by means of the screw itself) of such element on the femoral component 3 occurs. Simultaneously, a kind of “stop” is created that prevents the protrusion 25 from exiting from the space or opening in which it is inserted.

The pin 47, when present, can be made integrally with the body of the closure or locking element 46 or it can be assembled or fixed to the same according to known modes.

The closure or locking element 46 and/or the pin 47 can be made of a metallic material or of a plastic material such as polyethylene or ultra-high molecular weight polyethylene (UHMWPE).

When both the pin 47 and the closure or locking element 46 are made of plastic material, they can be glued or locked or joined in a stable manner to the femoral component 3, for example by means of the bone cement.

When the closure or locking element 46 and/or the pin 47 are made of plastic material, they can be glued or locked or joined in a stable manner to the femoral component 3, for example by means of bone cement.

The closure or locking element 46 has an internal face 46d (during use directed towards the bottom wall 35d of the box element 35) that is substantially concave. In such a manner, the sections 26′, 26″ can also rotate into such position of contact with the internal face 46d, and hence the femoral component 3 can also be articulated in this position with respect to the tibial component 2.

The closure or locking element 46 substantially has a prism shape, e.g. with triangular base, as visible in FIG. 22.

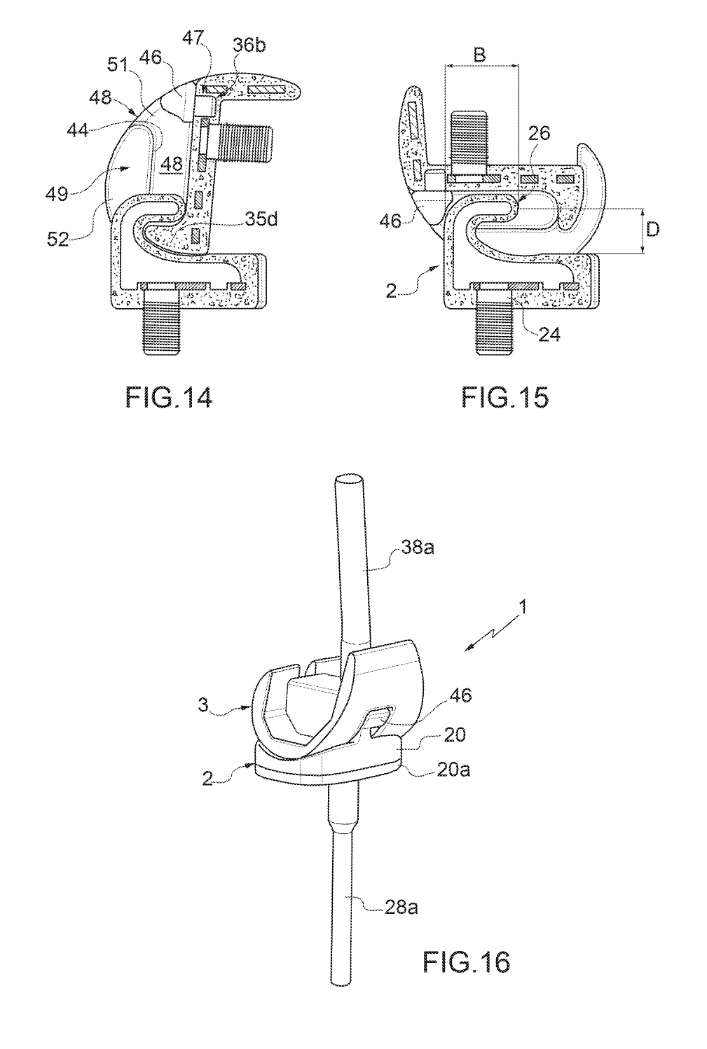

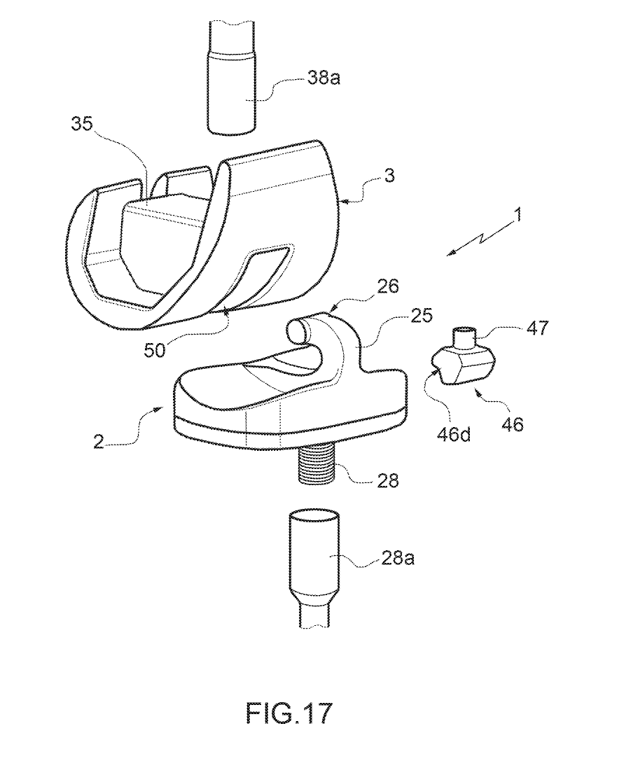

Seen in cross section, i.e. in a section parallel to the sagittal plane of the human body, as illustrated in the FIGS. 14 and 15, the first opening 48 has a substantially wedge-like shape while the second opening 49 has a progression that is substantially wedge-shaped or L-shaped. In the latter version, for example, the sections of the L interest (the longer one) the central portion towards the bottom and (the shorter one) the rear portion of the femoral component 3, at its external surface 32.

In particular, the second opening 49 is a through opening, from front to rear of the femoral element 3.

The first opening 48, instead, is only open at its front part. In the rear part, however, it is closed by the bottom wall 35d of the box element 35.

The bottom wall 35d, therefore, acts as a block for the sliding of the head or end 26 on the steps 44 and hence as a block for the maximum articulation of the femoral component 3 with respect to the tibial component 2.

The bottom wall 35d has a curvature substantially corresponding to that of the sections 26′, 26″ of the protrusion 25, in a manner so as to allow the rotation thereof—and hence the rotation of the femoral component 3 on the tibial component 2—also in such position.

The first and the second opening 48, 49 identify corresponding portions in the internal lateral walls of the box element 35.

Such portions are delimited, on each side, by the step 44.

In particular, the internal lateral walls have a first portion or surface 51 and a second portion or surface 52.

Both such portions have a progression substantially parallel to the sagittal plane of the human body. The first portion or surface 51, nevertheless, is placed more in proximity to the internal connector wall of the box element 35. In such a manner, it is situated more internally with respect thereto.

Such first portion or surface 51 has a progression that is substantially rectangular with smoothed edges. The height of such first portion or surface 51 is substantially equal to the bulk of each of the sections 26′, 26″.

The sections 26′ and 26″, in one version of the invention, are small cylinders which depart laterally from the protrusion 25. In a further version, the sections 26′, 26″ have a shape adapted to allow the rotation of the femoral component 3 on the tibial component 2, e.g. small balls, etcetera.

The sections 26′, 26″, in fact, act as a rotation hinge or axis around which a relative rotation occurs between the tibial component 2 and the femoral component 3. The second portion or surface 52 is instead more external with respect to the internal connector wall of the box element 35. In particular, such second portion or surface 52 is closer to the tibial component 2, with respect to the first 51.

This second portion or surface 52 has a substantially L-shaped progression, in which its larger section is substantially parallel to the progression of the first portion or surface 51, while its shorter section continues upward in a substantially perpendicular manner, considering the constrained prosthesis 1 during use.

The distance between the first portions or surfaces 51 is equal to L while the distance between the two second portions or surfaces 52 is equal to N.

The closure or locking element 46, at least in one version of the invention, determines the front wall of the box element 35.

As is seen, the present invention allows attaining the pre-established objects, since it allows a rotational-translational and stable movement of the femoral component on the tibial component, even in the presence of damaged bone tissues or bone tissues that have been weakened due to the size of the damage or repetition of the operations. Such movement cannot translate laterally, but only in front-back direction, since the femoral component is constrained in an articulated manner to the tibial component.

For such reason, therefore, the prosthesis 1 comprises means for the rotation-translation or for the sliding and the rotation of the femoral component 3 on the tibial component 2 and vice versa. The rotation-translation or rotation and sliding or translation movement is a relative movement of the femoral component 3 on the tibial component 2.

Such means, in at least one version of the invention, are constituted by the protrusion 25, with its head or end 26 and by the intercondylar opening or space 50 of the femoral component 3, possibly defined by the presence of the step 44.

The prosthesis according to the present invention, therefore, follows the physiological movement of the knee joint.

If necessary, the closure or locking element 46 could be removed from the prosthesis 1, for example in the remote case in which the latter must be removed from the human body, preserving the surrounding tissues as much as possible during the step of extraction of the various components.

The constrained prosthesis 1, according to a non-limiting version of the invention, is assembled according to the following method: arranging a tibial component 2 and a femoral component 3 as described above and inserting or housing the protrusion 25 of the tibial component inside an opening present in the femoral component 3.

In particular, the head or end 26 of the tibial component 2 is inserted in the first opening 48 of the femoral component 3. Then, the sections 26′, 26″ abut against a step 44 placed on both sides of the opening of the femoral component 3, and they can slide on the step itself.

In this manner, the protrusion 25 can slide and traverse the second opening 49 of the femoral component 3. Simultaneously, the latter is articulated with the tibial component by rotating and/or translating thereon.

Finally, the closure or locking element 46 is inserted in the femoral component 3, for example in a hole 36b thereof, in a manner so as to prevent the protrusion 25 from exiting from the femoral component 3. In such a manner, the femoral component 3 and the tibial component 2 are constrained to each other in an articulated manner. The at least one pin 28, 28a and/or the cylindrical protrusion or stem 38, 38a can then be inserted or screwed, in a manner so as to stabilize (during use) the application respectively of the tibial component 2 on the tibial bone and/or of the femoral component 3 on the femoral bone.

Finally, it is possible to lock the closure or locking element 46 by means of bone cement or by means of a screw.

If it is necessary to once again extract the prosthesis 1, it would be sufficient to remove the bone cement that blocks and fixes the closure or locking element 46 in position, e.g. by means of a small scalpel, so to be able to remove and free the passage for the protrusion 25.

Or, if there is the screw for locking the closure or locking element 46 to the femoral component 3: if it is necessary to once again extract the prosthesis 1, it would be sufficient to unscrew the screw that locks and fixes the closure or locking element 46 in position, so to be able to remove and free the passage for the protrusion 25.

By making the latter exit from the first opening 48, it will be possible to release the tibial and femoral components and then remove both components (or at least one) from the implant site without having to overly compromise the residual tissues of the articulation of the patient.

As seen above, the prosthesis 1—even if implanted in situations of serious bone loss and loosening of the ligaments as well as in less damaged situations—is constrained, thus ensuring good stability for the knee joint.

In addition, the prosthesis according to the present invention can, during use, be implanted in the two affected bone ends and be constrained after implant of the femoral and tibial components. Indeed, once the femoral component and tibial component are implanted, it is possible to insert the protrusion 25 in the suitable space or opening of the femoral component. The access point of the protrusion in the femoral element is then closed by the presence of the closure or locking element.

A facilitated method is thus obtained, with advantages in terms of reduction of time and pain, both for the patient and for the doctor.

In addition, due to this the patient can lead a self-sufficient life for the entire period of use of the constrained prosthesis according to the present invention.

The constrained prosthesis 1 according to the present invention is made of biologically compatible manner Such biologically compatible manner can be selected from among metals, metal alloys and organometallic compounds.

The constrained prosthesis according to the present invention, in fact, must be made of extremely durable materials, both since it is a permanent prosthesis, and since—being constrained—it must be able to resist stresses and forces of considerable intensity. Simultaneously, the material constituting the prosthesis according to the present invention must ensure a limited wear over time. This is facilitated by the fact that the tibial plate 20 is made of a plastic material with low friction coefficient, such as polyethylene or ultra-high molecular weight polyethylene (UHMWPE).

Therefore, while the femoral component 3 and/or the tibial base 20a can be made of a metallic material, among those listed above such as steel, chrome-cobalt steel, the tibial plate 20 can be made of a low friction plastic material, such as polyethylene material or UHMWPE.

The UHMWPE material is self-lubricating.

The thickness of the tibial plate 20, in one version of the invention, is comprised between 10 and 15 mm.

The thickness of the femoral component 3 or of the tibial base 20a, in one version of the invention, can be comprised between 1 and 5 mm, e g 2 mm.

If the tibial plate 20 is made of a plastic material, the protrusion 25 and/or the head 26 can be made of metal.

In one version of the invention, the protrusion 25 and/or the head 26 and/or the rib 23 are integrally made with the tibial base 20a, above which the tibial plate 20 made of plastic material is placed, as an insert. For example, the plastic material insert that constitutes the tibial plate 20 can only affect the zones relative to the two condylar articular bases 21, 22.

In such a manner, it is possible to confer a greater stability to the plant, a high load strength, etcetera.

The invention thus conceived is susceptible to numerous modifications and variations, all falling within the scope of the inventive concept.

In addition, all the details can be substituted by other technically equivalent elements. In practice, the materials used, as well as the contingent shapes and sizes, can be of any type in accordance with requirements, without departing from the protective scope of the following claims.

1. A prosthesis for the articulation of the knee comprising a tibial component, adapted to be fixed to one end of the tibial bone in proximity to the knee joint, and a femoral component, adapted to be fixed to one end of the femoral bone in proximity to the knee joint, wherein said tibial component is adapted to come into contact and be articulated with said femoral component, wherein said tibial component comprises a protrusion adapted to be inserted in an opening present in said femoral component, in order to allow the sliding and rotation of said femoral component on said tibial component, wherein said protrusion has a substantially C-shaped conformation.

2. The prosthesis according to claim 1, comprising means for the relative roto-translation of said femoral component on said tibial component, wherein said means for the roto-translation comprise at least said protrusion, and/or wherein said protrusion has a head or end provided with two sections which are extended on the side opposite said protrusion and/or said head or end.

3. The prosthesis according to claim 1, wherein said tibial component comprises a tibial plate provided with two condylar articular bases and a rib placed between said condylar articular bases, wherein said condylar articular bases are adapted to come into contact and be articulated with said femoral component.

4. The prosthesis according to claim 1, wherein said femoral component has a substantially U-shaped form in cross section according to a plane parallel to the sagittal plane of the human body and comprises a substantially concave internal surface, in contact with the femoral bone seat, and a substantially convex external surface adapted to come into contact with said tibial component.

5. The prosthesis according to claim 1, wherein said femoral component comprises a first and a second condylar portion, said first condylar portion being laterally placed and said second condylar portion being medially placed with respect to the sagittal plane of the human body, said condylar portions having a substantially U-shaped conformation in cross section according to a plane parallel to the sagittal plane of the human body, wherein said first condylar portion and said second condylar portion are separated from each other by an intercondylar space.

6. The prosthesis according to claim 4, of the preceding claims, wherein said femoral component comprises a box element, placed on the internal surface of said femoral component, wherein said box element comprises two lateral walls, a connector wall connected to said lateral walls, wherein said walls identify an internal connector wall and two internal lateral walls which delimit said opening or said intercondylar space.

7. The prosthesis according to claim 1, wherein said opening comprises a first opening having width (L) and a second opening having width (N), wherein (L) is greater than (N).

8. The prosthesis according to claim 2, wherein said a head or end provided with two sections has a width (F) and wherein said protrusion has a width (F1), wherein said width (F) substantially corresponds to or is slightly smaller than said width (L) and wherein said width (F1) substantially corresponds to or is slightly smaller than said width (N).

9. The prosthesis according to claim 6, wherein said means for the roto-translation comprise at least a step placed on each internal lateral wall of said box element, and/or each internal lateral wall of said box element comprises a step (44), wherein said step (44) delimits a first portion or surface and a second portion or surface of said internal lateral wall, wherein said first portion or surface is placed in proximity to the internal connector wall of the box element while said second portion or surface is placed in proximity to said tibial component.

10. The prosthesis according to claim 9, wherein said first portions or surfaces are separated by a distance (L) while the second portions or surfaces are separated by a distance (N) of said intercondylar space or of said respective first opening and said second opening.

11. The prosthesis according to claim 8, wherein said step (44) has an overturned S conformation and/or wherein said first opening (48) is delimited on the rear by a bottom wall (35d) of said box element (35).

12. The prosthesis according to claim 7, comprising a closure or locking element adapted to close said first opening and to prevent the exit of said protrusion from said opening of said femoral component, wherein said closure or locking element comprises a pin or a through hole for example for a locking screw.

13. The prosthesis according to claim 9, wherein said first portion or surface has a substantially rectangular progression with smoothed edges, and/or wherein said first portion or surface has a height substantially equal to the bulk of said sections.

14. The prosthesis according to claim 9, wherein said second portion or surface has a substantially L-shaped progression, wherein its greater segment is substantially parallel to the progression of said first portion or surface, while its shortest segment continues upward in a substantially perpendicular manner, considering the arrangement of said prosthesis during use.

15. The prosthesis according to claim 8, wherein said sections have a conformation adapted to allow the rotation of said femoral component on said tibial component and/or have a cylinder, ball conformation, or the like.

16. The prosthesis according to claim 1, wherein said prosthesis (1) is made of biologically compatible material selected from among a metal, a metal alloy, an organometallic compound.

17. The prosthesis according to claim 3, wherein said tibial component comprises a tibial base on which said tibial plate is abutted or fixed.

18. The prosthesis according to claim 17, wherein said tibial base is made of biologically compatible material selected from among a metal, metal alloy, an organometallic compound and/or wherein said tibial plate and/or said closure or locking element and/or said pin are made of a plastic material with low friction coefficient, such as polyethylene or ultra high molecular weight polyethylene.

19. A method for the assembly of a prosthesis for the articulation of the knee comprising a tibial component, adapted to be fixed to one end of the tibial bone in proximity to the knee joint, and a femoral component, adapted to be fixed to one end of the femoral bone in proximity to the knee joint, wherein said femoral component is adapted to come into contact and be articulated with said tibial component, comprising the steps of:
providing a tibial component, provided with a tibial plate and with a protrusion, and a femoral component, provided with an opening, wherein said protrusion has a substantially C-shaped conformation,
inserting said protrusion in said opening of said femoral component, in a manner such to constrain said femoral component to said tibial component in a rotary manner and allow the sliding and rotation of said femoral component on said tibial component.

20. The method according to claim 19, wherein said inserting step comprises inserting a head or end provided with two sections of said protrusion in a first opening having width (L).

21. The method according to claim 19, wherein said femoral component has a box element having two internal lateral walls and an internal connector wall, wherein each internal lateral wall comprises a step, with substantially overturned S conformation, wherein said method comprises the steps of sliding and/or rolling said sections on said step in a manner so as to allow the sliding and the rotation of said femoral component on said tibial component.

22. The method according to claim 20, comprising a step of providing a closure or locking element and closing said first opening by means of said closure or locking element, so as to prevent the exit of said protrusion from said opening of said femoral component.
